(19) United States
(12) Patent Application Publication
(10) Pub. No.: US 2008/0166423 A1
(43) Pub. Date: Jul. 10, 2008

(54) Combination Medication for Treating the Effects of Stomach Acid Reduction Medication on Bone Integrity

(76) Inventor: Renjit Sundharadas, San Diego, CA (US)

Correspondence Address: ABSOLUTE TECHNOLOGY LAW GROUP LLC, 135 W. WELLS ST., SUITE 518, MILWAUKEE, WI, 53203, US

(21) Appl. No.: 11/948,829

(22) Filed: Nov. 30, 2007

Related U.S. Application Data
(60) Provisional application No. 60/958,539, filed on Jul. 7, 2007.
(60) Provisional application No. 60/879,188, filed on Jan. 6, 2007.

(51) Int. Cl. A61K 33/42; A61K 31/59; A61K 31/19

(52) U.S. Cl. 424/602; 514/167; 514/338; 514/557

(57) ABSTRACT

A composition for addressing a calcium deficiency caused by chronic administration of proton pump inhibitors and other acid lowering agents comprised of at least one proton pump inhibitor and vitamin D. The composition can further include one or more of the following: at least one buffering agent in an amount sufficient to increase gastric fluid pH to a pH that prevents acid degradation of at least a portion of the proton pump inhibitor in the gastric fluid, a therapeutically effective amount of at least one non-steroidal anti-inflammatory drug, a therapeutically effective amount of at least one bisphosphonate or selective estrogen receptor modulator, and at least one calcium supplement.

### CROSS-REFERENCE TO RELATED APPLICATIONS

This application claims the benefit of and priority to the following U.S. provisional applications, incorporated herein in their entireties: provisional application Ser. No. 60/958,539, filed on 7 Jul. 2007 and provisional application Ser. No. 60/879,188, filed on 6 Jan. 2007.

### FIELD OF INVENTION

This invention relates generally to the field of medical treatment of gastrointestinal/cardiovascular ailments, and specifically to a combination medication for treating the effects of stomach acid reduction medication on bone integrity due to reduced calcium absorption and method of using same.

### BACKGROUND

Excessive gastric acid production can lead to gastrointestinal disorders such as heartburn, peptic ulcers, and gastric ulcers. Proton pump inhibitors (PPIs) decrease the production of gastric acid. Proton pump inhibitors (such as Nexium® and Protonix®) make up an almost fourteen billion dollar market, and this value does not include other acid suppression medications such as Zantac®, Tagamat®, Rolaids®, and Tums®.

However, gastric acid is necessary to break down food in the stomach, including breaking down calcium so that it can be absorbed. Thus, use of PPIs can lead to loss of bone mass, resulting in osteoporosis and breaking of hip bones. According to a recent study (see “Long-Term Proton Pump Inhibitor Therapy and Risk of Hip Fracture” JAMA Dec. 27, 2006 Vol. 296 No. 24 p. 2947), taking a proton pump inhibitor for more than one year resulted in an increase of hip fractores of 44% one year after therapy, and bone fractures increased commensurate with the length of the therapy. Hip fractures result in increased costs of medical expenditures and long term care/hospitization, vertegral compression, and bone fractures other than hips, and most importantly an increase of between 15% and 36% mortality rates after one year of treatment or more.

### DETAILED DESCRIPTION OF EMBODIMENTS OF THE INVENTION

For the purpose of promoting an understanding of the present invention, references are made in the text hereof to embodiments of a combination medication for treating the effects of stomach acid reduction medication on bone integrity and methods of using same, only some of which are described herein. It should nevertheless be understood that no limitations on the scope of the invention are thereby intended. One of ordinary skill in the art will readily appreciate that modifications such as the exact amount of the constituents, alternate but functionally similar components from which the combination medication is made, and the inclusion of additional constituents are deemed readily apparent and obvious to one of ordinary skill in the art, and all equivalent relationships to those described in the written description do not depart from the spirit and scope of the present invention. Some of these possible modifications are mentioned in the following description. Therefore, specific details disclosed herein are not to be interpreted as limiting, but rather as a basis for the claims and as a representative basis for teaching one of ordinary skill in the art to employ the present invention in virtually any appropriately detailed apparatus or manner.

Moreover, the term “substantially” or “approximately” as used herein may be applied to modify any quantitative representation that could permissibly vary without resulting in a change in the basic function to which it is related. For example, one embodiment of the combination medication is disclosed herein as including a daily dose of a proton pump inhibitor of between approximately five milligrams (5 mg) and approximately three hundred twenty milligrams (320 mg). The actual daily dose of the proton pump inhibitor may be outside of this range and still be within the scope of the invention if its efficacy is not materially altered.

The combination medication addresses calcium deficiency caused by chronic administration of proton pump inhibitors (PPIs) and other acid lowering agents. Thus the most basic example would be at least one proton pump inhibitor, and at least one calcium supplement and vitamin D. A similar combination would include at least one proton pump inhibitor, vitamin D, at least one calcium supplement, and at least one acid buffer to immediately reduce stomach acid and/or provide for a low acidic stomach environment so that any proton pump inhibitor in the combination would be immediate released. Further permutations allow the addition of at least one non-steroidal anti-inflammatory drug (NSAID), optionally at least one medication to reduce the cardiovascular side effects of NSAIDs to the original combination of at least one calcium supplement, at least one proton pump inhibitor, vitamin D, and zero, one or more acid buffers. Another use is to combine at least one bisphosphonate and/or at least one selective estrogen receptor modulator (e.g., Raloxifene) with at least one proton pump inhibitor, vitamin D, and zero one or more acid buffers.

It should be noted that the prior art does not address the need for calcium supplementation with proton pump inhibitors. This is proved by the fact that no combination medication includes Vitamin D combined with any calcium supplementation. Additionally, the combination involving at least on bisphosphonate and/or at least one selective estrogen receptor modulator is unique in using proton pump inhibitors and supplemental calcium to treat osteoporosis and its effects. Essentially, since osteoclasts, which reabsorb (destroy bone), have proton pumps in them, the proton pump inhibitor is thus used to decrease the function of the osteoclast. Then, the supplemental calcium is used to ensure that the acid lowering effect does not decrease the body's intake of calcium. Furthermore, since bisphosphonates often cause stomach discomfort secondary to increased stomach acid, the proton pump inhibitor reduces these symptoms and thus helps with patient compliance.

Of note, Calcium citrate would likely be absorbed more readily in the face of a low acidic environment produced by H2 blockers (and other chemicals that decrease acid production) or proton pump inhibitors. Calcium dosage would be sufficient to meet recommended guidelines while also taking into effect poor absorption states.

PPIs decrease stomach acidity. It is precisely this acidity that is responsible for ionizing and solubilizing insoluble calcium so that it can be absorbed and internalized by the body for multiple uses including maintaining bone integrity. Conversely, there are cells in the body called osteociasts that are involved in bone remodeling. These cells reabsorb (i.e., destroy) bone by using a proton pump which is actually also inhibited by the proton pump inhibitor (e.g., Nexium®). The fact that osteoclasts use proton pumps reinforces the importance of these pumps in solubilizing calcium.

There are essentially two forces at work: the PPI preventing calcium absorption in the gut by shutting off the proton pumps and decreasing acidity (promotes fractures) and the PPI inhibiting the proton pump in the osteoclasts which actually inhibits fractures by stopping these cells from dissolving bone. The main purpose of the December 2006 Journal of the American Medical Association (JAMA) study was to determine which effect dominated. The study showed increased hip fractures by forty-four percent (44%). Importantly, this effect was more severely correlated with higher doses, and most importantly, with the duration of PPI use. Thus, the study strongly supports the viewpoint that inhibiting the proton pump has a real cause-effect relationship that increases morbidity and mortality.

Additionally, the study involved 192,000 PPI users and 1.4 million acid suppression non-users (a clearly adequate sample size). It also considered age, sex, risks for falls, medications, and other comorbidities. The multivariate adjusted odds ratio (AOR) for all potential confounders was 1.44, or an increase in fractures of 44%. Table 2 of the study (found on page 2950 of Dec JAMA) shows the increases in fractures rates with time from 1 to 4 years. This increase in AOR was 1.59-1.22, or 0.37, or a thirty-seven percent (37%) increase with time for the maximum duration in the study of four (4) years. Table 3 of the study shows the increase in fractures with higher dosages. This is notable since all PPI products depend on higher dosing and prolonged duration of action for greater efficacy in reducing acid related symptoms and morbidity.

While the study was retrospective, a prospective study is simply not feasible. The study is essentially a gold standard study based on the sample size and that no drug company will spend millions of dollars to put tens of thousands of patients through a prospective trial, with the endpoint four (4) years out, that proves that their PPI causes significant side effects. For example, Vioxx® and Zelnorm® have proven that well-studied medications can be brought to market and yet have side effects that are unacceptable and, in the case of those two (2) medications, require removal from the marketplace.

Unlike Vioxx® and Zelnorm®, the side effects of which are cardiovascular and difficult to understand, the side effects of PPI (increased hip fractures) are much easier to understand and correct based on the knowledge of the mechanisms of calcium metabolism. The morbidity/mortality of hip fractures is great and since the correcting factor of calcium supplementation makes sense from a mechanism standpoint, it is the ideal candidate for a combination medication patent.

This combination medication would be a great product for Medicare part D recipients if it is sold to Medicare as a proprietary generic with the goal of matching generic pricing while taking market share amongst Medicare recipients. Since calcium absorption decreases with age and calcium excretion through the kidneys increases with age, it would benefit Medicare since their more vulnerable patients would be getting a safer medication which would reduce Medicare's longer term expenditures on hip fractures and other osteoporosis related diagnoses.

It should be noted that the combination medication of the immediate release formulation differs from the prior art in that the prior art include only a proton pump inhibitor in combination with an acid buffer. The acid buffer is said to include calcium citrate. Their acid suppression medication uses sodium bicarbonate for acid lowering. However, the combination medication of the immediate release formulation includes Vitamin D, since it is geared to promoting bone health and reducing the risk of osteoporosis and subsequent fractures.

The combination medication of the immediate release application can be combined in multiple combinations. In one embodiment, the combination medication can further include a stomach acid medication with calcium supplementation with the possibility of adding a buffering agent, as well as the possibility of an adjuvant for calcium supplementation absorption. In other embodiments, the combination medication further includes the addition of one or more NSAIDs and/or other pain medications as well as an anticoagulant for stroke and CV prophylaxis. Following are several non-limiting examples of various combinations of a therapeutically effective amount of at least one pump inhibitor, which may or may not be acid labile, a therapeutically effective amount of at least one calcium supplement agent including vitamin D, and one or more additional constituents. The one or more additional constituents can be selected from a group including one or more H2 blockers, one or more buffering agents, one or more substances that either help with calcium absorption/metabolism or with bone health, one or more NSAIDs, acetaminophen, and one or more cardiovascular prophylaxis agents.

It should be noted that any of the above constituents may be controlled/extended release and dosage ranges for any component may vary from one hundredth ( 1/100) one hundred (100) times the standard dose of each individual constituent. These doses are standard medical practice and are located in medical literature as well as package inserts accompanied by the medication. Certain components could be encapsulated as needed. For example, a standard proton pump inhibitor such as omeprazole is normally dosed at twenty to forty milligrams per day (20-40 mg/day). Thus one embodiment of the instant invention would cover 0.2 mg to 4,000 mg per day.

Examples of proton pump inhibitors include, but are not limited to TAK-390, AZD-0865, AR-H047108, CS-526, pumaprazole, revaprazan, soraprazan, esomeprazole, omeprazole, tenatoprazole, lansoprazole, rabeprazole, hydroxyomeprazole, dontoprazole, habeprazole, perprazole, ransoprazole, pantoprazole, pariprazole, leminoprazole and nepaprazole or a free base, a free acid, or a salt, hydrate, ester, amide, enantiomer, isomer, tautomer, polymorph, prodrug, or derivative of such compounds, and combinations thereof. The proton pump inhibitor is of Formula (I): wherein R.sup.1 is hydrogen, alkyl, halogen, cyano, carboxy, carboalkoxy, carboalkoxyalkyl, carbamoyl, carbamoylalkyl, hydroxy, alkoxy which is optionally fluorinated, hydroxyalkyl, trifluoromethyl, acyl, carbamoyloxy, nitro, acyloxy, aryl, aryloxy, alkylthio, or alkylsulfinyl; R.sup.2 is hydrogen, alkyl, acyl, acyloxy, alkoxy, amino, aralkyl, carboalkoxy, carbamoyl, alkylcarbamoyl, dialkylcarbamoyl, alkylcarbonylmethyl, alkoxycarbonylmethyl, or alkylsulfonyl; R.sup.3 and R.sup.5 are the same or different and each is hydrogen, alkyl, alkoxy, amino, or alkoxyalkoxy; R.sup.4 is hydrogen, alkyl, alkoxy which may optionally be fluorinated, or alkoxyalkoxy; Q is nitrogen, CH, or CR.sup.1; W is nitrogen, CH, or CR.sup.1; y is an integer of 0 through 4; and Z is nitrogen, CH, or CR.sup.1; or a free base, salt, ester, hydrate, amide, enantiomer, isomer, tautomer, prodrug, polymorph, or derivative thereof. Of note, the proton pump inhibitor dosages, while not limited to this amount, is usually between approximately five milligrams (5 mg) and approximately three hundred twenty milligrams (320 mg) per day.

One example of an H2 blocker (also known as H2 receptors or an H2 antagonist) is Ranitidine, which is normally dosed at a total of between approximately seventy five milligrams (75 mg) and approximately three hundred milligrams (300 mg) per day. Thus, in one embodiment of the combination medication in accordance with the present invention includes approximately 7.5 mg to approximately 30,000 mg per day (i.e., one hundredth ( 1/100) one hundred (100) times the standard dose). The H2 Blocker is selected from but not limited to the group consisting of Ranitidine, cimetidine, nizatidine, ranitidine, roxatidine, famotidine, ebrotidine, burimamide, metiamide, tiotidine, oxmetidine, pabutidine, lafutidine, nizatidine, pharmaceutically acceptable salts thereof, and combinations thereof. Of note, the blocker dosages, while not limited to, is typically between approximately five milligrams (5 mg) and approximately two thousand milligrams (2000 mg) per day.

For a buffering agent, such as sodium bicarbonate, the standard dose may contain between approximately three hundred twenty five milligrams (325 mg) and approximately two thousand milligrams (2000 mg) and can be taken every four (4) hours. Thus, one embodiment of the combination in accordance with the present invention includes between approximately three thousand twenty five hundred thousandths of a milligram (3.25 mg) and approximately two hundred thousand milligrams (200,000 mg), as often as every four (4) hours. Non-limiting examples of suitable buffering agents include sodium bicarbonate, aluminum, magnesium hydroxide, aluminum hydroxide/magnesium hydroxide co-precipitate, aluminum hydroxide/sodium bicarbonate co-precipitate, aluminum glycinate, calcium acetate, calcium bicarbonate, calcium borate, calcium carbonate, calcium citrate, calcium gluconate, calcium glycerophosphate, calcium hydroxide, calcium lactate, calcium phthalate, calcium phosphate, calcium succinate, calcium tartrate, dibasic sodium phosphate, dipotassium hydrogen phosphate, dipotassium phosphate, disodium hydrogen phosphate, disodium succinate, dry aluminum hydroxide gel, L-arginine, magnesium acetate, magnesium aluminate, magnesium borate, magnesium bicarbonate, magnesium carbonate, magnesium citrate, magnesium gluconate, magnesium hydroxide, magnesium lactate, magnesium metasilicate aluminate, magnesium oxide, magnesium phthalate, magnesium phosphate, magnesium silicate, magnesium succinate, magnesium tartrate, potassium acetate, potassium carbonate, potassium bicarbonate, potassium borate, potassium citrate, potassium metaphosphate, potassium phthalate, potassium phosphate, potassium polyphosphate, potassium pyrophosphate, potassium succinate, potassium tartrate, sodium acetate, sodium bicarbonate, sodium borate, sodium carbonate, sodium citrate, sodium gluconate, sodium hydrogen phosphate, sodium hydroxide, sodium lactate, sodium phthalate, sodium phosphate, sodium polyphosphate, sodium pyrophosphate, sodium sesquicarbonate, sodium succinate, sodium tartrate, sodium tripolyphosphate, synthetic hydrotalcite, tetrapotassium pyrophosphate, tetrasodium pyrophosphate, tripotassium phosphate, trisodium phosphate, trometarnol, and combinations thereof (based in part upon the list provided in The Merck Index, Merck & Co. Rahway, N.J. (2001)). In addition, due to the ability of proteins or protein hydrolysates to react with stomach acids, they too can serve as buffering agents in the present invention. Furthermore, combinations or mixtures of the above mentioned buffering agents can be used in the pharmaceutical formulations described herein. Of note the buffering agent is not limited to but usually ranges between approximately two hundred milligrams (200 mg) and approximately five thousand milligrams (5000 mg). Additionally the range can also be between approximately 0.1 mEq to approximately five thousand (5000) mEq.

Calcium supplementation, such as by calcium citrate, has a normal dose of between approximately two hundred milligrams (200 mg) and approximately five hundred milligrams (500 mg) per dose and can often be taken up to four (4) times per day. Thus, one embodiment of the combination in accordance with the present invention includes between approximately two milligrams (2 mg) and approximately fifty thousand milligrams (50,000 mg) per dose with the option of being taken as many as four (4) times per day. The calcium component may include, but is not be limited to, calcium carbonate and calcium citrate, which are the most popular supplement types. Others such as calcium gluconate, calcium citrate malate, calcium phosphate, calcium lactate, and calcium from dolomite (an extract from limestone and marble that also contains magnesium) may also be components, including combinations thereof. Of note, calcium citrate would likely be absorbed more readily in the face of a low acidic environment produced by H2 blockers or proton pump inhibitors. Calcium citrate may be in the colloid form. Calcium would preferentially but not absolutely dissolve within forty (40) minutes of ingestion Dosage would be sufficient to meet recommended guidelines while also taking into effect poor absorption state.

For an agent such as vitamin D, the normal dose is between approximately one hundred twenty five (125) units and approximately two hundred (200) units per dose and, as above, can be taken up to four (4) times per day. Thus, one embodiment of the combination in accordance with the present invention includes between approximately one and a quarter units (1.25 IU) and approximately twenty thousand units (20,000 IU) per dose, and as many as four (4) doses per day. The Vitamin D component may be selected from, but not limited to Vitamin D, cholecalciferol, ergocalciferol, Vitamin D1, Vitamin D2, Vitamin D3, Vitamin D4, Vitamin D5, 7-dehydrocholesterol, 25-hydroxycholecalciferolMagnesium, Zinc, Fluoride, Manganese, Copper, vitamin C, vitamin k, lactose, boron, and combinations thereof. Vitamin D intake usually should not exceed one thousand (1000) IU daily (to avoid toxicity) except in an impaired absorption situation. Dosages would be in accordance to known guidelines.

An NSAID, such as ibuprofen, would have a standard dose ranging from approximately two hundred milligrams (200 mg) to approximately eight hundred milligrams (800 mg), taken as many as four (4) times per day. There are many different NSAIDs each with their own dose ranges that widely vary and are well documented. Thus, one embodiment of the combination in accordance with the present invention includes between approximately two milligrams (2 mg) and eighty thousand milligrams (80,000 mg) per dose, with up to four (4) doses per day. The NSAID is selected from, but not limited to, the group including ibuprofen, salicylates, indomethacin, flurbiprofen, diclofenac, ketorolac, naproxen, piroxicam, tebufelone, ibuprofen, flurbiprofen, etodolac, nabumetone, tenidap, alcofenac, antipyrine, aminopyrine, dipyrone, aminopyrone, phenylbutazone, oxaprozin, clofezone, oxyphenbutazone, prexazone, apazone, benzydamine, bucolome, cinchopen, clonixin, ditrazol, epirizole, fenoprofen, Flurbiprofen, floctafeninl, flufenamic acid, glaphenine, indoprofen, ketoprofen, meclofenamic acid, mefenamic acid, niflumic acid, phenacetin, salidifamides, sulindac, suprofen, tolmetin, pharmaceutically acceptable salts thereof, and combinations thereof. The NSAID is also defined as including the group of COX-2 inhibitors. A composition in which the COX-2 inhibitor is a compound or a pharmaceutically acceptable salt thereof, or any hydrate thereof selected from but not limited to oxyphenbutazone; azapropazone; phenylbutazone, rofecoxib, etoricoxib, lumiracoxib, celecoxib (Celebrex), valdecoxib, parecoxib, Vioxx, meloxicam, droxicam, lomoxicam, tenoxicam, or a 5-alkyl-2-arylaminophenylacetic acid derivative COX-2 inhibitor, e.g. COX189. This group of NSAIDs is inclusive of COX-2 inhibitors known by trade name as Arcoxia, Dynastat, and Prexige. NSAID may also include CS-502, JTE-522, L-745,337, or NS398. JTE-522, L-745,337 and NS398 as described, inter alia, in Wakatani, et al. (Jpn. J. Pharmacol. 78:365-371 (1998)) and Panara, et al. (Br. J. Pharmacol. 116:2429-2434 (1995)). The NSAID may include any NSAID combined with and antiulcer agent that is a prostaglandin selected from the group including misoprostol, PGE.sub.1, PGA.sub.1, PGB.sub.1, PGF.sub.1.alpha., 19-hydroxy-PGA.sub.1, 19-hydroxy-PGB.sub.1, PGE.sub.2, PGA.sub.2, PGB.sub.2, 19-hydroxy-PGA.sub.2, 19-hydroxy-PGB.sub.2, PGE.sub.3, PGF.sub.2.alpha., PGF.sub.3.alpha., PGI.sub.2, pharmaceutically acceptable salts, isomers and derivatives thereof, and combinations thereof. Additionally, for completeness, the NSAID may be selected from the group consisting of salicylates, indomethacin, flurbiprofen, diclofenac, ketorolac, naproxen, piroxicam, tebufelone, ibuprofen, etodolac, nabumetone, tenidap, alcofenac, antipyrine, aminopyrine, dipyrone, aminopyrone, phenylbutazone, clofezone, oxyphenbutazone, prexazone, apazone, benzydamine, bucolome, cinchopen, clonixin, ditrazol, epirizole, fenoprofen, floctafeninl, flufenamic acid, glaphenine, indoprofen, ketoprofen, meclofenamic acid, mefenamic acid, niflumic acid, phenacetin, salidifamides, sulindac, suprofen, tolmetin, pharmaceutically acceptable salts, isomers and derivatives thereof, and combinations thereof.

Acetaminophen has a standard dose ranging from between approximately three hundred twenty five milligrams (325 mg) to approximately one thousand milligrams (1000 mg) with some of the three hundred twenty five milligram (325 mg) or six hundred fifty milligram (650 mg) doses being taken up to between four (4) and six (6) times per day. Thus, one embodiment of the combination in accordance with the present invention includes between approximately three and one quarter mg (3.25 mg) and approximately one hundred thousand milligrams (100,000 mg) per dose, up to four (4) to six (6) doses per day. With this medication, a daily dosage above approximately four thousand milligrams (4000 mg) per day has known liver toxicity, so this would have to be considered when arriving at a final dose per tablet/capsule.

Aspirin is one (1) example of a cardiovascular prophylaxis agent and has a standard dose range of between approximately eighty one milligrams (81 mg) per day and approximately six hundred fifty milligrams (650 mg) every four (4) hours. Thus, one embodiment of the combination in accordance with the present invention includes between approximately eighty one hundredths of a milligram (0.81 mg) and approximately sixty five thousand milligrams (65,000 mg) per dose, with up to doses every four (4) hours per day. The cardiovascular prophylaxis agent is selected from the group consisting of aspirin, clopidogrel, ticlopidine, aggrenox, dipyridamole, cilostazol, trental, or salt thereof. However, the cardiovascular prophylaxis agents may also include, HMG-CoA reductase inhibitors, ACE inhibitors, angiotensin receptor blockers, calcium channel blockers, and any other vasodilatory or anti-hypertensive agents or platelet aggregation inhibitors, and oral anticoagulants. The group of HMG-CoA reductase inhibitors, ACE inhibitors, angiotensin receptor blockers, calcium channel blockers and any other vasodilatory or anti hypertensive agents, platelet aggregation inhibitors or oral anticoagulants is to be selected from, but not limited to: benazepril, captopril, enalapril, fosinopril, lisinopril, moexipril, perindopril, quinapril, ramipril, trandolapril, candesartan, eprosartan, ibesartan, losartan, telmisartan, valsartan, atorvastatin, cerivastatin, fluvastatin, lovastatin, pravastatin, simvastatin, aggrenox, dipyridamole, amlodipine, felodipine, isradipine, nicardipine, nifedipine, nisoldipine, diltiazem, verapamil, isosorbide dinitrate/mononitrate, combination of the aforementioned, or salt thereof.

In addition, the combination may further include one or more excipients, including, but not limited to one or more parietal cell activators, erosion facilitators, flavoring agents, sweetening agents, diffusion facilitators, antioxidants and carrier materials selected from one or more binders, suspending agents, disintegration agents, filling agents, surfactants, solubilizers, stabilizers, lubricants, wetting agents, diluents, anti-adherents, antifoaming agents, and combinations thereof.

Any component of the composition medication, or multiple components up to and including all components, can further include a gastric resistant coating, a controlled-release coating, an enzymatic-controlled coating, a film coating, a sustained-release coating, an immediate-release coating, and a delayed-release coating. In one embodiment in which the proton pump inhibitor is combined with a buffer, the proton pump inhibitor is non-enteric coated.

Following are some non-limiting, exemplary combinations in accordance with the present invention:

### EXAMPLE 1

A proton pump inhibitor, such as omeprazole, combined with CaCO₃, calcium citrate, and Vitamin D. For one (1) day dosing, this could be between approximately twenty milligrams (20 mg) and approximately forty milligrams (40 mg) of omeprazole, one thousand milligrams (1000 mg) of calcium carbonate, five hundred milligrams (500 mg) calcium citrate, and two hundred (200) IU of Vitamin D. Such a combination is particularly applicable as a product for mass consumption since the proton pump inhibitors are taken by many people and by lowering their stomach acid it may inhibit calcium absorption. Variations on this idea include making part of the proton pump inhibitor longer release possibly by encapsulating it so that the proton pump inhibitor has both a long-acting and short-acting component for true twenty four hour coverage. The calcium citrate is added since it is absorbed more readily in a lower acidity environment. The calcium citrate could also be delayed release so that it is absorbed better over time. The Vitamin D is to aid in bone homeostasis. The CaCO₃ can be for calcium supplementation as well as to rapidly reduce stomach acidity so that the omeprazole may act more immediately.

### EXAMPLE 2

An NSAID, aspirin, proton pump inhibitor, CaCO₃, calcium citrate, and Vitamin D. This would be a pain medication that provides gastrointestinal and cardiovascular prophylaxis from the NSAID while also assisting with calcium supplementation since calcium absorption may be suppressed due to the low stomach acidity caused by the proton pump inhibitor. As with the previously presented example, some constituents of this example could be delayed/extended release.

### EXAMPLE 3

Another possible combination is an H2 blocker combined with calcium citrate and Vitamin D. This could likely be dosed twice per day and thus provide better calcium absorption since it is being dosed more than once per day. Sample dosages could be one hundred fifty milligrams (150 mg) ranitidine, five hundred milligrams (500 mg) calcium citrate, and two hundred (200) IU of vitamin D.

### EXAMPLE 4

Another possible combination is a proton pump inhibitor combined with calcium citrate and Vitamin D. For example, twenty milligrams (20 mg) omeprazole, five hundred milligrams (500 mg) calcium citrate, and two hundred (200) IU of Vitamin D, which would address the chronic proton pump inhibitor patient's risk of calcium deficiency. As above, the calcium citrate could be extended release for better absorption.

### EXAMPLE 5

A generic proton pump inhibitor (e.g., omeprazole), sodium bicarbonate, and calcium citrate with Vitamin D for the immediate release combination. For a delayed release combination, the combination would include the generic proton pump inhibitor (e.g., omeprazole) and calcium citrate with Vitamin D. However, because a sizable amount of calcium may be needed for adequate supplementation that the patient may need to take 2 or more smaller pills for convenience.

As provided supra, because any component may be extended release, the calcium may be released in that manner to achieve better absorption. Calcium supplementation such as calcium citrate may be high dose such as one thousand milligrams (1000 mg) or more per tablet to meet the body's daily requirement. Additionally a study would likely be very favorable (regarding bone homeostasis) to such a product since the proton pump inhibitor patient with calcium supplementation would be actually getting calcium supplementation compared to the baseline proton pump inhibitor patient who by taking the medication may actually have a long term reduction in calcium absorption.

It should be understood that the previous examples are intended to be non-limiting, as many combinations of the medication exist.

Overall, any composition may be a tablet, a suspension tablet, a bite suspension tablet, a bite-disintegration tablet, a rapid dispersion tablet, a rapid disintegration tablet, a chewable tablet, an effervescent tablet, a bilayer tablet, a caplet, a capsule, a powder, a lozenge, a sachet, a cachet, a troche, a pellet, a granule, a microgranule, a powder, an effervescent powder, and an aqueous suspension produced from powder, and combinations thereof. Any component above may be sustained, immediate, or delayed release, quickly dissolving, delayed dissolving, sequentially dissolving, or have an enteric coating in order to alter the dissolving rate. One example of this would be combining omeprazole that is either immediate release or normal release with an extended release omeprazole so twenty four hour protection is maintained. For instance, omeprazole combined with sodium bicarbonate is more of an immediate release formulation lasting only eighteen (18) hours. The addition of a sustained release omeprazole would provide full twenty four hour coverage. When addressing decreased calcium absorption cause by medications that lower stomach acidity, this medication is advantageous because patients are likely to take their stomach acid medication to avoid discomfort but less motivated to buy and take calcium as supplementation.

Moreover, as will be readily understood by one of ordinary skill in the art, no individual component should be given in such a high dose as to inhibit calcium absorption/utilization as well as normal bone physiology.

While the composition for treating the effects of stomach acid reduction medication on bone integrity has been shown and described with respect to several embodiments and uses in accordance with the present invention, it is to be understood that the same is not limited thereto, but is susceptible to numerous changes and modifications as known to a person of ordinary skill in the art, and it is intended that the present invention not be limited to the details shown and described herein, but rather cover all such changes and modifications obvious to one of ordinary skill in the art.

It is important to realize the the above discussion of dosing includes extremes such as dosages that are clearly either toxic or subtherapeutice. These harmful doses would never be in a formulation that reaches the market. However, wide claims have been made regarding dosage range in order to clearly include all therapeutic dose ranges.

Furthermore, bisphosphonates and selective estrogen receptor modulators are known to have common dose ranges. These range anywhere from five milligrams (5 mg) to one hundred fifty milligrams (150 mg) so acceptable dose ranges could range from 0.5 mg to 500 mg.

Further possible combinations could include a proton pump inhibitor, an optional buffer, optional vitamin D, optional calcium supplementation, and a prokinetic agent or sleep aid.

What is claimed is:

1. A composition for treating the effects of stomach acid reduction medication on bone integrity comprised of:
a therapeutically effective amount of at least one proton pump inhibitor; and
a therapeutically effective amount of vitamin D.

2. The composition of claim 1, wherein said at least one proton pump inhibitor is selected from a group consisting of TAK-390, AZD-0865, AR-H047108, CS-526, pumaprazole, revaprazan, soraprazan, esomeprazole, omeprazole, tenatoprazole, lansoprazole, rabeprazole, hydroxyomeprazole, dontoprazole, habeprazole, perprazole, ransoprazole, pantoprazole, pariprazole, leminoprazole, nepaprazole, and a free base, a free acid, a salt, an hydrate, an ester, an amide, an enantiomer, an isomer, a tautomer, a polymorph, a prodrug, a derivative of said group, and combinations thereof.

3. The composition of claim 1 further including at least one calcium supplement, said at least one calcium supplement selected from a group consisting of calcium carbonate, calcium citrate, calcium gluconate, calcium citrate malate, calcium phosphate, calcium lactate, calcium from dolomite, and combinations thereof.

4. The composition of claim 1, further including at least one excipients selected from a group consisting of at least one parietal cell activators, at least one erosion facilitator, at least one flavoring agent, at least one sweetening agent, at least one diffusion facilitators, at least one antioxidant, and at least one carrier material selected from at least one binder, at least one suspending agent, at least one disintegration agent, at least one filling agent, at least one surfactant, at least one solubilizer, at least one stabilizer, at least one lubricant, at least one wetting agent, at least one diluent, at least one anti-adherent, at least one antifoaming agent, and combinations thereof.

5. The composition of claim 1, wherein said vitamin D is selected from a group consisting of Vitamin D2, Vitamin D3, Vitamin D1, Vitamin D4, Vitamin D5, a supplement containing Vitamin D, a multivitamin containing Vitamin D, at least one precursor selected from a group consisting of 7-dehydrocholesterol, 25-hydroxycholecalciferol, cholecalciferol, ergocalciferol, Magnesium, Zinc, Fluoride, Manganese, Copper, vitamin C, vitamin k, lactose, boron, and combinations thereof.

6. The composition of claim 1, wherein said composition further includes at least one non-steroidal anti-inflammatory drug, said at least one non-steroidal anti-inflammatory drug selected from a group consisting of ibuprofen, a salicylate, indomethacin, flurbiprofen, diclofenac, ketorolac, naproxen, piroxicam, tebufelone, flurbiprofen, etodolac, nabumetone, tenidap, alcofenac, antipyrine, aminopyrine, dipyrone, aminopyrone, phenylbutazone, oxaprozin, clofezone, oxyphenbutazone, prexazone, apazone, benzydamine, bucolome, cinchopen, clonixin, ditrazol, epirizole, fenoprofen, Flurbiprofen, floctafeninl, flufenamic acid, glaphenine, indoprofen, ketoprofen, meclofenamic acid, mefenamic acid, niflumic acid, phenacetin, salidifamides, sulindac, suprofen, tolmetin, pharmaceutically acceptable salts thereof, and combinations thereof.

7. The composition of claim 1, wherein said composition further includes at least one bisphosphonate or estrogen receptor modulator selected from a group consisting of pamidronate, aledronate, ibandronate, zoledronate, risedronate, raloxifene, a free base, a free acid, or a salt, a hydrate, an ester, an amide, an enantiomer, an isomer, a tautomer, a polymorph, a prodrug, a derivative of said group, and combinations thereof.

8. A pharmaceutical composition comprising:
a therapeutically effective amount of at least one proton pump inhibitor;
a therapeutically effective amount of vitamin D; and
at least one buffering agent in an amount sufficient to increase gastric fluid pH to a pH that prevents acid degradation of at least a portion of said at least one proton pump inhibitor in said gastric fluid.

9. The pharmaceutical composition of claim 8, wherein said at least one proton pump inhibitor is selected from a group consisting of TAK-390, AZD-0865, AR-H047108, CS-526, pumaprazole, revaprazan, soraprazan, esomeprazole, omeprazole, tenatoprazole, lansoprazole, rabeprazole, hydroxyomeprazole, dontoprazole, habeprazole, perprazole, ransoprazole, pantoprazole, pariprazole, leminoprazole, nepaprazole, a free base, a free acid, a salt, a hydrate, an ester, an amide, an enantiomer, an isomer, a tautomer, a polymorph, a prodrug, a derivative of said group, and combinations thereof.

10. The pharmaceutical composition of claim 8, wherein said at least one buffering agent is selected from a group consisting of aluminum, magnesium hydroxide, aluminum hydroxide/magnesium hydroxide co-precipitate, aluminum hydroxide/sodium bicarbonate co-precipitate, aluminum glycinate, calcium acetate, calcium bicarbonate, calcium borate, calcium carbonate, calcium citrate, calcium gluconate, calcium glycerophosphate, calcium hydroxide, calcium lactate, calcium phthalate, calcium phosphate, calcium succinate, calcium tartrate, dibasic sodium phosphate, dipotassium hydrogen phosphate, dipotassium phosphate, disodium hydrogen phosphate, disodium succinate, dry aluminum hydroxide gel, L-arginine, magnesium acetate, magnesium aluminate, magnesium borate, magnesium bicarbonate, magnesium carbonate, magnesium citrate, magnesium gluconate, magnesium hydroxide, magnesium lactate, magnesium metasilicate aluminate, magnesium oxide, magnesium phthalate, magnesium phosphate, magnesium silicate, magnesium succinate, magnesium tartrate, potassium acetate, potassium carbonate, potassium bicarbonate, potassium borate, potassium citrate, potassium metaphosphate, potassium phthalate, potassium phosphate, potassium polyphosphate, potassium pyrophosphate, potassium succinate, potassium tartrate, sodium acetate, sodium bicarbonate, sodium borate, sodium carbonate, sodium citrate, sodium gluconate, sodium hydrogen phosphate, sodium hydroxide, sodium lactate, sodium phthalate, sodium phosphate, sodium polyphosphate, sodium pyrophosphate, sodium sesquicarbonate, sodium succinate, sodium tartrate, sodium tripolyphosphate, synthetic hydrotalcite, tetrapotassium pyrophosphate, tetrasodium pyrophosphate, tripotassium phosphate, trisodium phosphate, trometarnol, and combinations thereof.

11. The pharmaceutical composition of claim 8, further including at least one calcium supplement, said at least one calcium supplement selected from a group consisting of calcium carbonate, calcium citrate, calcium gluconate, calcium citrate malate, calcium phosphate, calcium lactate, calcium from dolomite, and combinations thereof.

12. The pharmaceutical composition of claim 8, further comprising at least one excipient selected from a group consisting of at least one parietal cell activator, at least one erosion facilitator, at least one flavoring agent, at least one sweetening agent, at least one diffusion facilitator, at least one antioxidant, at least one carrier material selected from binders, at least one suspending agent, at least one disintegration agent, at least one filling agent, at least one surfactant, at least one solubilizer, at least one stabilizers, at least one lubricant, at least one wetting agent, at least one diluent, at least one anti-adherent, at least one antifoaming agent, and combinations thereof.

13. The pharmaceutical composition of claim 8, wherein said vitamin D is selected from a group consisting of Vitamin D2, Vitamin D3, Vitamin D1, Vitamin D4, Vitamin D5, a supplement containing vitamin D, a multivitamin containing vitamin D, at least one precursor selected from a group consisting of 7-dehydrocholesterol, 25-hydroxycholecalciferol, cholecalciferol, ergocalciferol, Magnesium, Zinc, Fluoride, Manganese, Copper, vitamin C, vitamin k, lactose, boron, and combinations thereof.

14. The pharmaceutical composition of claim 8, wherein said pharmaceutical composition further includes at least one non-steroidal anti-inflammatory drug, said at least one non-steroidal anti-inflammatory drug selected from a group consisting of ibuprofen, a salicylate, indomethacin, flurbiprofen, diclofenac, ketorolac, naproxen, piroxicam, tebufelone, flurbiprofen, etodolac, nabumetone, tenidap, alcofenac, antipyrine, aminopyrine, dipyrone, aminopyrone, phenylbutazone, oxaprozin, clofezone, oxyphenbutazone, prexazone, apazone, benzydamine, bucolome, cinchopen, clonixin, ditrazol, epirizole, fenoprofen, Flurbiprofen, floctafenini, flufenamic acid, glaphenine, indoprofen, ketoprofen, meclofenamic acid, mefenamic acid, niflumic acid, phenacetin, salidifamides, sulindac, suprofen, tolmetin, pharmaceutically acceptable salts thereof, and combinations thereof.

15. The pharmaceutical composition of claim 8, wherein said pharmaceutical composition further includes at least one bisphosphonate or estrogen receptor modulator selected from a group consisting of pamidronate, aledronate, ibandronate, zoledronate, risedronate, raloxifene, a free base, a free acid, a salt, a hydrate, an ester, an amide, an enantiomer, an isomer, a tautomer, a polymorph, a prodrug, a derivative of said group, and combinations thereof.

16. A combination medication for treating osteoporosis or osteopenia comprised of:
a therapeutically effective amount of at least one proton pump inhibitor;
a therapeutically effective amount of vitamin D; and
a therapeutically effective amount of at least one bisphosphonate or at least one selective estrogen receptor.

17. The combination medication of claim 16, wherein said at least one proton pump inhibitor is selected from a group consisting of TAK-390, AZD-0865, AR-H047108, CS-526, pumaprazole, revaprazan, soraprazan, esomeprazole, omeprazole, tenatoprazole, lansoprazole, rabeprazole, hydroxyomeprazole, dontoprazole, habeprazole, perprazole, ransoprazole, pantoprazole, pariprazole, leminoprazole, nepaprazole, and a free base, a free acid, a salt, a hydrate, an ester, an amide, an enantiomer, an isomer, a tautomer, a polymorph, a prodrug, a derivative of said group, and combinations thereof.

18. The combination medication of claim 16, wherein said pharmaceutical composition further includes at least one calcium supplement, said at least one calcium supplement selected from a group consisting of calcium carbonate, calcium citrate, calcium gluconate, calcium citrate malate, calcium phosphate, calcium lactate, calcium from dolomite, and combinations thereof.

19. The combination medication of claim 16, wherein said at least one bisphosphonate or at least one selective estrogen receptor modulator is selected from a group consisting of pamidronate, aledronate, ibandronate, zoledronate, risedronate, raloxifene, a free base, a free acid, a salt, a hydrate, an ester, an amide, an enantiomer, an isomer, a tautomer, a polymorph, a prodrug, a derivative of said group, and combinations thereof.

20. The combination medication of claim 16, further comprising at least one excipient selected from a group consisting of at least one parietal cell activator, at least one erosion facilitator, at least one flavoring agent, at least one sweetening agent, at least one diffusion facilitator, at least one antioxidant, at least one carrier material selected from at least one binder, at least one suspending agent, at least one disintegration agent, at least one filling agent, at least one surfactant, at least one solubilizer, at least one stabilizer, at least one lubricant, at least one wetting agent, at least one diluent, at least one anti-adherent, at least one antifoaming agent, and combinations thereof.

21. The combination medication of claim 16, wherein said vitamin D is selected from a group consisting of Vitamin D2, Vitamin D3, Vitamin D1, Vitamin D4, Vitamin D5, a supplement containing vitamin D, a multivitamin containing vitamin D, at least one precursor selected from a group consisting of 7-dehydrocholesterol, 25-hydroxycholecalciferol, cholecalciferol, ergocalciferol, Magnesium, Zinc, Fluoride, Manganese, Copper, vitamin C, vitamin k, lactose, boron, and combinations thereof.

22. The combination medication of claim 16, wherein said combination medication further includes at least one buffering agent, said at least one buffering agent selected from a group consisting of aluminum, magnesium hydroxide, aluminum hydroxide/magnesium hydroxide co-precipitate, aluminum hydroxide/sodium bicarbonate co-precipitate, aluminum glycinate, calcium acetate, calcium bicarbonate, calcium borate, calcium carbonate, calcium citrate, calcium gluconate, calcium glycerophosphate, calcium hydroxide, calcium lactate, calcium phthalate, calcium phosphate, calcium succinate, calcium tartrate, dibasic sodium phosphate, dipotassium hydrogen phosphate, dipotassium phosphate, disodium hydrogen phosphate, disodium succinate, dry aluminum hydroxide gel, L-arginine, magnesium acetate, magnesium aluminate, magnesium borate, magnesium bicarbonate, magnesium carbonate, magnesium citrate, magnesium gluconate, magnesium hydroxide, magnesium lactate, magnesium metasilicate aluminate, magnesium oxide, magnesium phthalate, magnesium phosphate, magnesium silicate, magnesium succinate, magnesium tartrate, potassium acetate, potassium carbonate, potassium bicarbonate, potassium borate, potassium citrate, potassium metaphosphate, potassium phthalate, potassium phosphate, potassium polyphosphate, potassium pyrophosphate, potassium succinate, potassium tartrate, sodium acetate, sodium bicarbonate, sodium borate, sodium carbonate, sodium citrate, sodium gluconate, sodium hydrogen phosphate, sodium hydroxide, sodium lactate, sodium phthalate, sodium phosphate, sodium polyphosphate, sodium pyrophosphate, sodium sesquicarbonate, sodium succinate, sodium tartrate, sodium tripolyphosphate, synthetic hydrotalcite, tetrapotassium pyrophosphate, tetrasodium pyrophosphate, tripotassium phosphate, trisodium phosphate, trometarnol, and combinations thereof.
